(19) United States
(12) Patent Application Publication
(10) Pub. No.: US 2024/0057890 A1
(43) Pub. Date: Feb. 22, 2024

(54) NON-INVASIVE DETERMINATION OF BLOOD GLUCOSE LEVELS

(71) Applicant: Roche Diabetes Care, Inc., Indianapolis, IN (US)

(72) Inventors: Jeffrey M. Bender, Fishers, IN (US); Herbert Fink, Mannheim (DE); Tim Maihoefer, Mannheim (DE); Jochen Schulat, Mannheim (DE)

(21) Appl. No.: 18/500,815

(22) Filed: Nov. 2, 2023

Related U.S. Application Data
(63) Continuation of application No. PCT/EP2022/061681, filed on May 2, 2022.
(60) Provisional application No. 63/183,674, filed on May 4, 2021.

(30) Foreign Application Priority Data
May 21, 2021 (EP) 21 175 372.8

(51) Int. Cl. A61B 5/08; G01N 33/497; A61B 5/097; A61B 5/00

(52) U.S. Cl. CPC A61B 5/082; G01N 33/497; A61B 5/097; A61B 5/7271; G01N 2033/4975

(57) ABSTRACT

A method to determine a subject's blood glucose level non-invasively by analyzing an exhaled breath or another gaseous emanation from the subject. The method includes non-invasively detecting an amount of at least one volatile organic marker in the subject's exhaled breath or the subject's other gaseous emanation as marker data and determining the subject's blood glucose level based on the marker data. The amount of the volatile organic marker selected is negatively correlated to the blood glucose level.

### RELATED APPLICATIONS

This application is a continuation of International Application Serial No. PCT/EP2022/061681, filed May 2, 2022, which claims priority to U.S. Patent Application Ser. No. 63/183,674, filed May 4, 2021, and which also claims priority to European Patent Application Serial No. 21 175 372.8, filed May 21, 2021, the entire disclosures of all of which are hereby incorporated herein by reference.

### BACKGROUND

This disclosure relates generally to a non-invasive determination of a subject's blood glucose level based on detecting volatile organic markers in exhaled breath or other gaseous emanation from the subject.

The monitoring of blood glucose levels is crucial for diabetes management. A typical glucose measurement method involves piercing the skin, typically a finger, to draw blood and applying the blood to a chemically active disposable medium. To avoid the necessity of repeatedly piercing the skin, different non-invasive blood glucose monitoring technologies have also been proposed.

An example of a non-invasive blood glucose monitoring method is disclosed in WO 2020/02989 A1, the method combining infrared measurement with simultaneous pressure reading. Still, with infrared spectroscopy it is difficult to meet an in-vivo accuracy required for a reliable blood glucose management.

Another example of a non-invasive blood glucose monitoring technology is described in U.S. Pat. No. 7,076,371 B2. A volatile marker, which is characteristic of the disease, is detected in exhaled breath or other gaseous emanation from the person and the detected marker data is analyzed in an artificial neural network with a fuzzy filter. For determining blood glucose levels, the markers are selected to measure the destruction or deterioration of cell membranes by lipid peroxidation or protein oxidation, e.g., propanol or acetone, as those substances from metabolic processes of intestinal bacteria are positively correlated to blood glucose levels.

In Trefz, P., Obermeier, J., Lehbrink, R. et at. “Exhaled volatile substances in children suffering from type 1 diabetes mellitus: results from a cross-sectional study.” Scientific Report 9, 15707 (2019), https://doi.org/10.1038/s41598-019-52165-x, the measurements for the determination of blood glucose levels are focused on the exhalation of ethanol, acetone, isopropanol, dimethyl sulfide, isoprene, pentanal, and limonene as these compounds have been previously associated to the disturbed glucose homeostasis or reflect a metabolic link to diabetes type 1 related comorbidities, dyslipidemia, and oxidative stress.

Ire Rydosz, Artur: “A Negative Correlation Between Blood Glucose and Acetone Measured in Healthy and Type 1 Diabetes Mellitus Patient Breath,” JOURNAL OF DIABETES SCIENCE AND TECHNOLOGY, Vol. 9, No. 4, 17 Feb. 2015 (2015-02-17), pages 881-884, XP055856949, US ISSN: 1932-2968, DOI: 10.1177/1932296815572366 a negative correlation between an acetone concentration in exhaled breath and a blood glucose level is reported.

In Van den Velde, S. et al.: “GC-MS analysis of breath odor compounds in liver patients,” JOURNAL OF CHROMATOGRAPHY B, ELSEVIER, AMSTERDAM, NL, Vol. 875, No. 2, 15 Nov. 2008 (2008-11-15), pages 344-348, XP025879919, ISSN: 1570-0232, DOI: 1016/J .JCHROMB.2008.08.031 a decreased concentration of Indole in a subject's exhaled breath was identified as sign for a liver disease.

An example of a device for non-invasive blood glucose monitoring is a skin surface sampling system as described in U.S. Pat. No. 10,143,447 B2 entitled “Skin surface sampling system.” This system utilizes an elongated collection tube with a sampling head positioned on one end in contact with the patient's skin. A liquid supply absorbs volatile organic compounds (VOC) and semi-volatile organic compounds (SVOC from the surface of the skin and collects the mixed liquid in a sample collection device. The system is operated by positioning the sampling head on the skin surface and then flushing the liquid supply through a set of channel grooves in the sampling head that direct the mixed liquid to the collection tube. Although providing a potentially non-invasive form of blood glucose level monitoring, the system requires a liquid capture system.

### SUMMARY

Despite of the advantages and the progress achieved by the above-mentioned developments, some significant technical challenges remain regarding responsiveness and reliability. This disclosure provides a fast responding and robust blood glucose monitoring method.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

In a first aspect of this disclosure, a method to determine a subject's blood glucose level non-invasively by analyzing an exhaled breath or another gaseous emanation from the subject is disclosed. The method comprises non-invasively detecting an amount of at least one volatile organic marker in the subject's exhaled breath or the subject's other gaseous emanation as marker data and determining the subject's blood glucose level based on the marker data. The volatile organic marker is selected from a group of markers for which the amount of the volatile organic marker is negatively correlated to the blood glucose level.

A volatile organic marker, also called volatile organic compound (VOC), is an organic chemical that has a high vapor pressure or rather a low boiling point. Thus, VOCs are volatile at rather low temperatures such as room temperature for example. The human body is a major source of VOCs that originate from different parts and processes within the body. So-called endogenous VOCs originate from metabolic processes within the body. An important organ involved in generating and transforming such endogenous VOCs is the liver. VOCs originating from the environment are called exogenous VOCs, e.g., entering the body via respiratory air, food intake or diffusion through skin or originating from the microbiome, e.g., of the digestive tract.

VOCs generated or absorbed in various parts of the body enter the bloodstream and pass into the exhaled breath by gas exchange in the lungs. VOCs from skin originate from secretion of eccrine, sebaceous or apocrine glands.

It is noted, that a VOC may as well represent a derivative of a compound of interest, i.e., a compound originating from an endogenous process, or a fragment of a compound of interest, said fragment being formed by cleavage of the molecule during the analytical process.

Different methods for detecting an amount of a VOC or of a multitude of VOCs in a sample of exhaled breath or of gaseous emanation are known. According to a first embodiment, the volatile organic marker is detected via mass spectrometry, i.e., measuring the so-called mass-to-charge ratio (m/z) of the ionized compounds. In a further embodiment, the compounds are detected using proton transfer reaction time of flight mass spectrometry (PTR-ToF-MS) which comprises chemical ionization of the sample's compounds based on proton transfer. Typically, H₃O⁺ ions are used for protonation of VOCs with adequate proton affinity. The PTR-ToF-MS enables a direct analysis without sample preparation and with a high sensitivity as well as a fast response.

Alternative detection methods to detect a volatile organic marker in real-time are Selected Ion Flow Tube Mass Spectrometry (SIFT-MS) or secondary electrospray ionization high-resolution mass spectrometry (SESI-HRMS).

The sample of exhaled breath is captured using a mouthpiece, a nasal cannula, a handheld breath analyzer or any other device suitable to capture at least a fraction of the exhaled breath.

To detect an amount of one or more volatile organic marker from other gaseous emanation a sample of the secretion of one or more parts of the body is captured, e.g., from the head, chest, back, armpit, waist, arm or genital area.

It is understood that the detected amount of each volatile organic marker comprises a single value or a multitude of values over time wherein each value represents a single measurement or an average over two or more measurements. It is also understood that the amount of the volatile organic marker is either determined in absolute values or in relative values, e.g., determining an amount of change of the marker concentration.

The person's blood glucose level may be determined from the detected amount of one or more volatile organic markers in the exhaled breath or other emanation due to the correlation between said entities. It is understood that the correlation provides for values of blood glucose derived from absolute or relative values of the volatile organic marker concentration in the person's breath.

According to an embodiment, the marker data is normalized based on a reference signal, e.g., derived from ambient air or from another marker in the same sample of exhaled breath or from another sample of the person's breath or other emanation. In a further embodiment the set-up to perform the method is taught and/or trained based on individual metabolic processes, e.g., creating individual profiles for a person/patient. For example, the training involves selecting the volatile organic marker from a group of volatile organic markers also comprising derivatives or fragments of endogenous compounds.

Surprisingly, a negative correlation between the volatile organic marker amount and the blood glucose level has proven to be particularly beneficial for the reliability, responsiveness and sensitivity of the method.

The negative correlation may only be achieved by compounds that do have a functional role in the insulin response. The amount of such compounds, i.e., markers, in breath or other emanation are essentially independent of external influences or individual characteristics of the patient.

According to an embodiment of this disclosure, the volatile organic marker is one of indole (C₈H₇N), a partly saturated derivative of indole, a fully saturated derivative of indole and a true fraction of indole.

indole is an aromatic heterocyclic organic compound with formula C₈H₇N. It is widely distributed in the natural environment and can be produced by a variety of bacteria, such as E. coli, which are usually present in the human intestine. Furthermore, indole is the most abundant metabolite produced from digestion of tryptophan. As many bacteria present in the human intestine can synthesize indole from tryptophan due to the enzyme tryptophanase the indole level in the human intestine is constant.

Moreover, indole is a possible signaling molecule for stimulation of Glucagon-Like-Protein-1 (GLP-1), which is needed for an accurate insulin response. In the presence of glucose, indole diffuses into intestinal cells and increases GLP-1 secretion by blocking K⁺ channels. This results in a decrease of indole concentration in the extracellular space, a decrease that is then observed in breath. As soon as extracellular glucose levels drop, indole diffuses out of cells again and detaches from K⁺ channels. These reversible mechanisms indicate that indole acts as a signal molecule only and is not metabolized. A result is the inverse progression of indole compared to blood glucose levels, i.e., the negative correlation. Furthermore, said correlation enables a monitoring of the blood glucose levels in real-time as any change in the blood glucose concentration occurs simultaneously to a change of the amount of Indole. “Simultaneously” here means that the times at which a change in the two concentrations occurs are separated by a maximum of five minutes.

A derivative is a compound that is derived from a similar compound by a chemical reaction. Some derivatives of indole, e.g., aliphatic C8-amines like cyclohexyl-ethylamine (C₈H₁₈N) or octylamine (C₈H₁₇N) or isomers thereof show a corresponding correlation to blood glucose as well as the described advantage. This also holds for some fragments, e.g., benzenes, wherein fragments denominate products of fragmentation, i.e., the dissociation of energetically unstable molecular ions formed from passing the molecules in the ionization chamber of a mass spectrometer. The term “true fragment” is used to indicate that said fragment originates from the dissociation of the claimed compound, i.e., indole.

Indole has a molecular weight of ˜117.1 g/mol. Thus, when detecting Indole via PTR-ToF-MS the mass-to-charge ratio after proton transfer for PTR-ToF-MS is m/z=118.1 due to the additional H+.

Derivatives are, for example, cyclohexyl-ethylamine C₈H₁₇N with a mass-to-charge ration of m/z=128.14 after protonation with H⁺ or octylamine C₈H₁₉N with a mass-to-charge reaction of m/z=130.15 after protonation.

Fragments of indole are, for example, benzene C₆H₆ with a mass-to-charge ration of m/z=79.055 after protonation with H⁺and C₇H₈ with a mass-to-charge ration of m/z=93.069 after protonation.

According to another embodiment, the method further comprises to detect at least one additional volatile organic marker non-invasively in the exhaled breath or the other gaseous emanation from the subject as additional marker data and to determine the subject's blood glucose level based on the marker data and the additional marker data, wherein the amount of the additional volatile organic marker and the blood glucose level are a positively correlated.

Pursuant to alternative embodiments, the additional volatile organic marker is one or more of carbon dioxide, nitric oxide, formaldehyde, propanol, propanoic acid, acetone, acetic acid, butanol, butyric acid, phenol and caprolactam. Alternatively, the additional volatile organic marker is a fragment or a derivative of one or more of carbon dioxide, nitric oxide, formaldehyde, propanol, propanoic acid, acetone, acetic acid, butanol, butyric acid, phenol and caprolactam. Although the additional marker data may not be as accurate and reliable, they do provide additional information. In an embodiment, additional marker data of a specific additional volatile organic marker is used to indicate hypo- or hyperglycemia additionally to the determination of the blood glucose level.

According to a further embodiment, the method further comprises non-invasively detecting an amount of at least one volatile organic marker in the subject's exhaled breath as well as in the subject's other gaseous emanation as marker data. Either the same volatile organic markers or different volatile organic markers in the breath and. the other gaseous emanation, respectively, are detected.

According to another embodiment, the method comprises the following steps: continuously monitoring the subject's breathing over a period of time and non-invasively detecting the amount of the at least one volatile organic marker in the continuously monitored subject's breathing over time as the maker data. Furthermore, temporal alterations of an amount of at least one control marker are detected non-invasively in the continuously monitored subject's breathing over time as control marker data. A temporal correlation between the marker data and the control marker data is determined and at least one segment of the marker data over time that corresponds to a temporal period of exhalation is selected based on the temporal correlation. The subject's blood glucose level is determined based on the selected segment of the marker data. Pursuant to alternative embodiments the control marker is one of O₂, CO, CO₂, NO, N₂, and H₂O.

According to an alternative embodiment, the method further comprises selecting at least one additional segment of the marker data over time based on the temporal correlation, wherein the additional segment corresponds to a temporal period of inhalation. A blank value is determined based on the additional segment and subtracted from the marker data before determining the subject's blood glucose level.

As breathing is a cyclic process, the control marker enables monitoring said chronological sequence and to distinguish periods of exhalation and periods of inhalation. The correlation of the control data with the marker data enables selecting one or more segments of the marker data corresponding to a desired period of the breathing cycles. While the exhaled breath contains VOCs originating from endogenous processes, the gas sample derived from temporal periods correlated to inhalation provides background information and, e.g., is used to derive a blank value.

in an alternative embodiment, to derive a blank value, the method comprises detecting an amount of at least one volatile organic marker in a gaseous reference sample non-invasively as reference data, determining a blank value based on the reference data and subtracting the blank value from the marker data before determining the subject's blood glucose level. Pursuant to a further embodiment, the gaseous reference sample contains ambient air.

According to a further embodiment, a non-invasive blood glucose level monitoring system for executing the method of any of the preceding embodiments comprises a volatile organic marker detecting system configured to detect an amount of the volatile organic marker in a sample as marker data, a vapor distribution member configured to bring a sample of exhaled breath or other gaseous emanation from a subject in contact with the volatile organic marker detecting system, and a processing unit configured to determine the subject's blood glucose level based on the marker data.

These and other advantages, effects, features and objects of the inventive concept will become better understood from the description that follows.

The advantages, effects, features and objects other than those set forth above will become more readily apparent when consideration is given to the detailed description below. In the description, reference is made to the accompanying drawings, which form a part hereof and in which there is shown by way of illustration, not limitation, embodiments of the inventive concept. The embodiments are depicted schematically and corresponding reference characters indicate corresponding parts throughout the several views of the drawings.

It should be understood, however, that the description of exemplary embodiments that follows is not intended to limit the inventive concept to the particular forms disclosed, but on the contrary, the intention is to cover advantages, effects, features and objects failing within the spirit and scope thereof as defined by the claims below. In particular, different embodiments may be realized in an isolated fashion as well as in any arbitrary feasible combination, as the skilled person will realize.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a flow chart of the inventive method to determine a subject's blood glucose level non-invasively by analyzing exhaled breath or another gaseous emanation from the subject;

FIG. 2 shows a schematic cross-section of a proton transfer reaction time of flight mass spectrometer;

FIG. 3 shows a correlation between a person's blood glucose level and the amount of Indole in the person's exhaled breath after glucose intake;

FIG. 4 shows a correlation between the blood glucose level and the amount of different VOCs in the exhaled breath after glucose intake;

FIG. 5 shows a correlation between the blood glucose level and the amount of different VOCs in the exhaled breath after lactulose intake and

FIG. 6 shows a correlation between the blood glucose level and the amount of Indole in the exhaled breath after lactulose intake.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

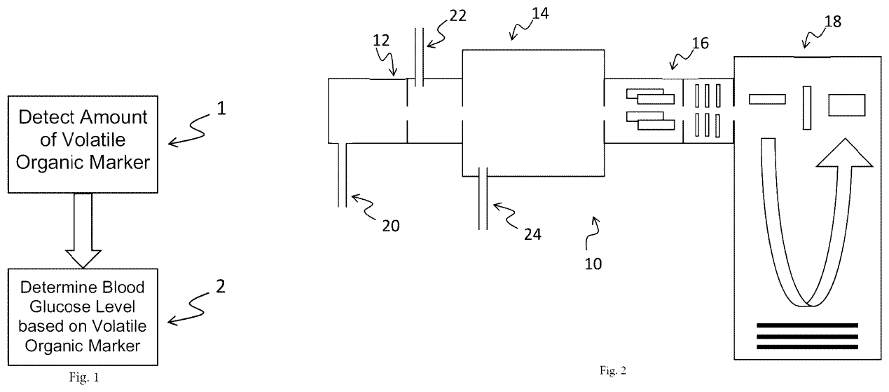

FIG. 1 depicts a flow chart of a method to determine a subject's blood glucose level non-invasively by analyzing an exhaled breath or another gaseous emanation from the subject. The method comprising at least a first step 1 of non-invasively detecting an amount of at least one volatile organic marker in the subject's exhaled breath or the subject's other gaseous emanation as marker data and a second step 2 of determining the subject's blood glucose level based on the marker data. The at least one volatile marker detected in step 1 is selected from a group of markers for which the amount of the volatile organic marker is negatively correlated to the blood glucose level.

FIG. 2 shows a schematic cross-section of a proton transfer reaction time of flight mass spectrometer (PTR-ToF-MS) 10 as one example of a device suitable to carry out step 1, i.e., to detect the amount of different VOCs in a sample, such as exhaled breath.

The shown mass spectrometer 10 consists of an ion source 12, a drift tube 14, a quadrupole ion guide 16 and a reflectron time of flight mass analyzer 18. Water vapor enters the ion source 12 through a water vapor inlet 20 and hydronium ions (H₃O⁺) are generated from the water vapor by a hollow cathode discharge. The ion source 12 further comprises an excess water vapor outlet 22. In the drift tube 14, the hydronium ions react in a proton transfer reaction with analytes, like the volatile organic marker to be detected, from a sample, i.e., exhaled breath of the subject that is injected through a sample gas inlet 24. In the depicted embodiment, said protonated analytes are directed towards the mass analyzer 18 through a two-part quadrupole ion guide 16. In the reflectron time of flight mass analyzer 18 the ionized analytes are accelerated such that all ions have the same kinetic energy and can be separated according to their mass to charge ration m/z via a time of flight measurement.

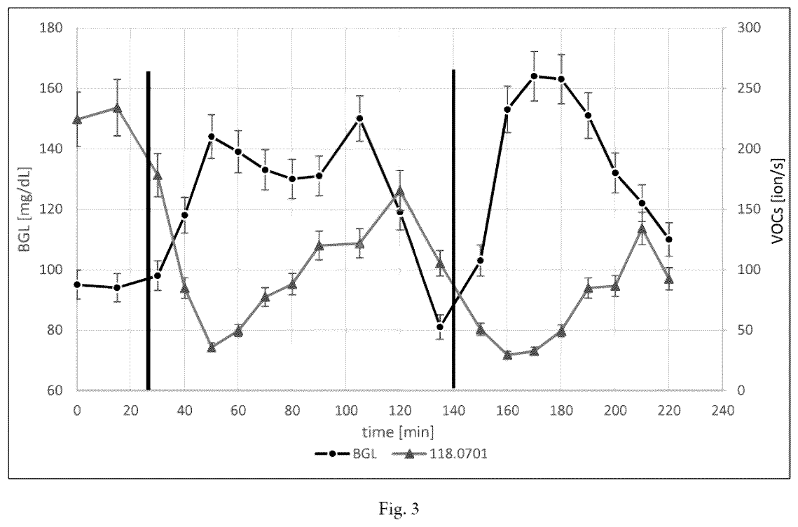

In FIG. 3, a graph is depicted showing a person's blood glucose levels BGL over time as well as the amount of Indole VOC in the person's exhaled breath over time. Over the depicted period of time the person started in a fasting state (no food intake during previous eight hours) with a blood glucose level BGL of about 90 mg/dL and ingested predefined amounts of glucose at two different points in time indicated by the vertical lines.

After each glucose intake (indicated by vertical lines), the blood glucose level BGL rises sharply to a peak value before starting to decrease again. Parallel to the increase of the blood glucose level BGL the amount of Indole VOC in the person's breath decreases and with the decrease of the blood glucose level BGL the amount of Indole VOC starts to rise again. Thus, the blood glucose level BGL and the amount of Indole VOC are negatively correlated.

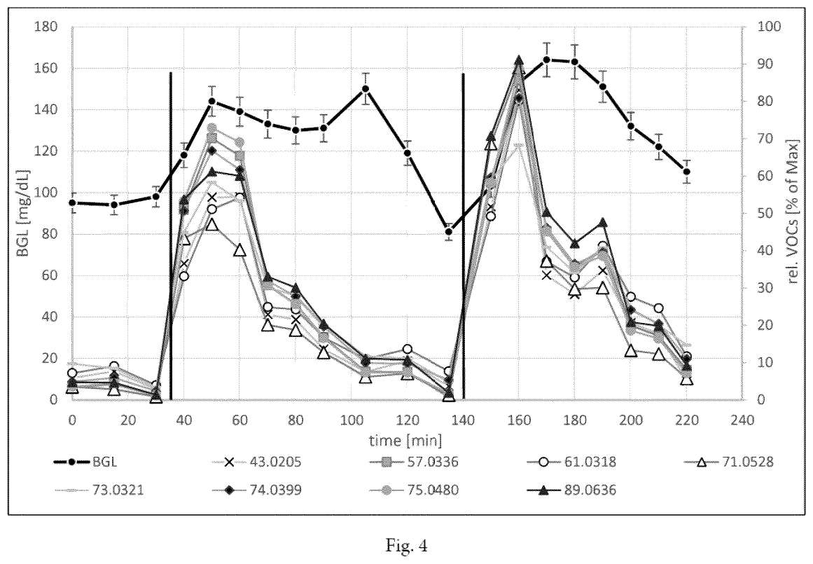

In FIG. 4, a graph is depicted showing the person's blood glucose levels BGL over time as in FIG. 3 and the amount of other volatile organic markers VOCs in the person's exhaled breath over time.

Unlike Indole, the other volatile organic markers VOCs shown in this graph exhibit a positive correlation to the rise of the blood glucose level BGL. Moreover, the decrease of said other volatile organic markers occurs significantly earlier in time than the decrease of the blood glucose level.

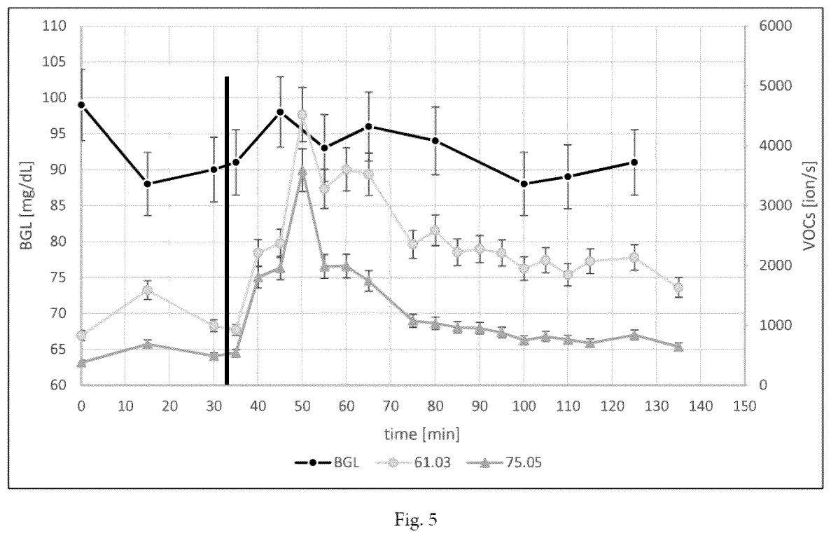

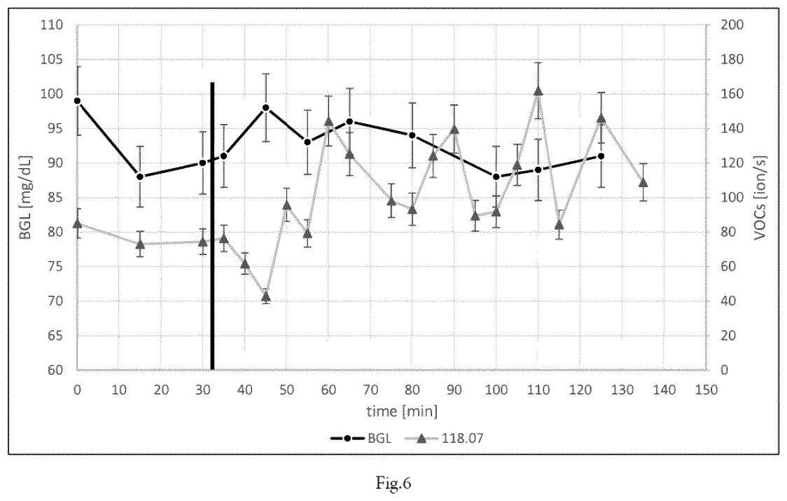

FIGS. 5 and 6 depict the correlation between the blood glucose level and the amount of different volatile organic compounds in the exhaled breath after lactulose intake. Lactulose is a disaccharide consisting of D-galactose and fructose and is not metabolized, i.e., digested, by the human organism. Instead, intestinal bacteria metabolize it. Correspondingly, the blood glucose level BGL is not affected by the intake of lactulose but stays constant around 95 mg/dL. A vertical line indicates the event of the lactulose intake in the figures.

As can be seen in FIG. 5, the other volatile organic markers VOCs do show a correlation, i.e., a considerable increase correlated to the event of the lactulose intake. Thus, these volatile organic markers are at least in parts originating from gut bacteria metabolism- and not directly correlated to the blood glucose metabolism. Therefore, the amount of these other organic markers is not optimal for blood glucose monitoring as it may be affected by external influences.

However, the amount of Indole VOC in the person's breath is not affected by the lactulose intake, i.e., it remains constant like the glucose level, as can be seen in FIG. 6. Thus, indole is not linked to a gut bacteria metabolism but to the glucose metabolism, and as such a suitable biomarker for glucose monitoring, untainted with external influences.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method of determining a subject's blood glucose level non-invasively by analyzing an exhaled breath or another gaseous emanation from the subject, comprising:
non-invasively detecting an amount of at least one volatile organic marker in the subject's exhaled breath or the subject's other gaseous emanation as marker data;
determining the subject's blood glucose level based on the marker data;
wherein the volatile organic marker is selected from a group of markers for which the amount of the volatile organic marker is negatively correlated to the blood glucose level; and
wherein the volatile organic marker is one of indole (C8H7N), a partly saturated derivative of indole, fully saturated derivative of indole and a true fraction of indole.

2. The method of claim 1, wherein the amount of the volatile organic marker is detected by mass spectrometry.

3. The method of claim 1, further comprising:
non-invasively detecting at least one additional volatile organic marker in the exhaled breath or the other gaseous emanation from the subject as additional marker data;
determining the subject's blood glucose level based on the marker data and the additional marker data;
wherein the additional volatile organic marker is selected from a group of markers for which the amount of the volatile organic marker is positively correlated to the blood glucose level.

4. The method of claim 3, wherein the additional volatile organic marker is one or more of carbon dioxide, nitric oxide, formaldehyde, propanol, propanoic acid, acetone, acetic acid, butanol, butyric acid, phenol and caprolactam.

5. The method of claim 3, wherein the additional volatile organic marker is a derivative or a fragment of one or more of carbon dioxide, nitric oxide, formaldehyde, propanol, propanoic acid, acetone, acetic acid, butanol, butyric acid, phenol and caprolactam.

6. The method of claim 1, further comprising non-invasively detecting an amount of at least one volatile organic marker in the subject's exhaled breath as well as in the subject's other gaseous emanation as marker data.

7. The method of claim 1, further comprising:
continuously monitoring the subject's breathing over a period of time;
non-invasively detecting the amount of the at least one volatile organic marker in the continuously monitored subject's breathing over time as the maker data;
non-invasively detecting temporal alterations of an amount of at least one control marker in the continuously monitored subject's breathing over time as control marker data;
determining a temporal correlation between the marker data and the control marker data;
selecting at least one segment of the marker data over time corresponding to a temporal period of exhalation based on the temporal correlation; and
determining the subject's blood glucose level based on the selected segment of the marker data.

8. The method of claim 7, wherein the control marker is one of O₂, CO, CO₂, N₂, and H₂O.

9. The method of claim 7, wherein the method further comprises:
selecting at least one additional segment of the marker data over time corresponding to a temporal period of inhalation based on the temporal correlation;
determining a blank value based on the additional segment; and
subtracting the blank value from the marker data before determining the subject's blood glucose level.

10. The method of claim 1, wherein the method further comprises:
non-invasively detecting an amount of at least one volatile organic marker in a gaseous reference sample as reference data;
determining a. blank value based on the reference data; and
subtracting the blank value from the marker data before determining the subject's blood glucose level.

11. A non-invasive blood glucose level monitoring system for executing the method of claim 1, comprising:
a volatile organic marker detecting system configured to detect an amount of the volatile organic marker in a sample as marker data;
a vapor distribution member configured to bring a sample of exhaled breath or other gaseous emanation from a subject in contact with the volatile organic marker detecting system; and
a processing unit configured to determine the subject's blood glucose level based on the marker data.
